(19) United States
(12) Patent Application Publication
(10) Pub. No.: US 2012/0330106 A1
(43) Pub. Date: Dec. 27, 2012

(54) THORACIC RETRACTORS AND METHODS

(75) Inventors: John T.M. Wright, (US); Woodrow G. Mathison, Denver, CO (US); Woodrow G. Mathison, Centennial, CO (US)

(73) Assignee: Genesee Biomedical, Inc., Denver, CO (US)

(21) Appl. No.: 13/532,386

(22) Filed: Jun. 25, 2012

Related U.S. Application Data
(60) Provisional application No. 61/532,679, filed on Sep. 9, 2011.
(60) Provisional application No. 61/500,697, filed on Jun. 24, 2011.

(51) Int. Cl. A61B 1/32

(52) U.S. Cl. 600/218

(57) ABSTRACT

A surgical thoracic retractor has retraction members that grip the anterior and posterior surfaces of the ribs between vice-like jaws to prevent a crushing or other force being applied to the intercostal nerves, thus minimizing the patient's post-operative pain.

### CROSS REFERENCE TO RELATED APPLICATIONS

This application is a continuation-in-part and claims the benefit of U.S. Provisional Application Nos. 61/532,679, filed Sep. 9, 2011 and 61/500,697, filed Jun. 24, 2011, the complete disclosures of which are herein incorporated by reference, including all references that are incorporated into each of such provisional applications.

### TECHNICAL FIELD

This invention relates generally to surgical instruments and methods for thoracic surgery. In particular, the invention relates to such instruments and methods to distance adjacent ribs without affecting or crushing the intercostal and other nerves.

### BACKGROUND OF THE INVENTION

A thoracotomy causes the patient much post-operative chronic pain, defined as pain after 6 months, is common and can occur in up to 67% of patients. The source of pain is thought to be crushing of the intercostal nerve by the force applied by the thoracic retractor used to create an intercostal space to allow surgical access to the chest cavity. The intercostal nerves extend along the lower margin of the ribs in the sub costal grooves below the intercostal arteries. According to Cerfolio R. J., Price T. N., Bryant A. S., Sale Bass C. and Bartolucci A. A., Intracostal Sutures Decease the Pain of Thoracotomy, Ann Thorac Surg 2003; 76:407-12, thoracic surgeons spend much time in managing patients' postoperative pain. Cerfolio et al. describes the use of intercostal sutures as well as a non-divided intercostal muscle flap to decrease pain. As another approach, D'Andrilli A., Ibrahim M., Ciccone A. M., et al., Intrapleural Intercostal Nerve Block Associated with Mini-thoracotomy Improves Pain Control After Major Lung Resection, Eur J Cardiothor Surg 2006; 29:790-4 describes the use of an intercostal nerve block to improve pain control.

Conventional thoracic retractors employ vertical lipped blades that are placed in the intercostal space after the intercostal muscles are divided. The blades are moved apart, thus pressing on the lower lip of the upper rib, and the upper face of the lower rib. Thus the upper blade causes compression and possible crushing of the intercostal nerve leading to severe postoperative pain.

This invention is directed towards overcoming one or more of the problems discussed above.

### BRIEF SUMMARY OF THE INVENTION

One embodiment provides a surgical thoracic retractor having retraction members that grip the anterior and posterior surfaces of the ribs between vice-like jaws to prevent any crushing or other forces being applied to the intercostal nerves, thus minimizing the patient's post-operative pain. The retraction members remain spaced apart from the intercostal nerves as the ribs are separated, thus preventing contact with the nerves during the surgical procedure.

In some cases, a distancing mechanism may be used to move the jaws apart. The distancing mechanism may include one or more joints to permit a wide range of movement of the jaws relative to the distancing mechanism as the ribs are being separated.

In one optional aspect, one or more guide members may be closely positioned relative to the jaws. These guide members have a through hole through which a drill bit may be placed to drill holes through the ribs. This permits pins to be placed through the ribs to further help stabilize the jaws. Also, following a procedure, the pins may be removed and a length of suture may be placed through the holes to facilitate closing the surgical site.

### BRIEF DESCRIPTION OF THE DRAWINGS

FIG. 1 illustrates a vertical section through an intercostal space, showing the relationship of the vessels and nerves to the muscular layers.

FIG. 2 is a plan view of one embodiment of a retractor of the invention with the retraction members in the closed position.

FIG. 3 is a partial sectional end view taken along line A-A of FIG. 2.

FIG. 4 is an end view of a retraction blade of the retractor of FIG. 2.

FIG. 5 is a side view of a retraction blade in the direction of arrow B of FIG. 4.

FIG. 6 is a side view of a retraction blade in the direction of arrow C of FIG. 4.

FIG. 7 is a part plan view of a pair of retraction blades of FIG. 1 shown partially rotated.

FIG. 8 is a cross-sectional view taken along line A-A of FIG. 2 with the ribs positioned between the jaws of the retractor blades.

FIG. 9 is a cross-sectional view taken along line A-A of FIG. 2 with the ribs positioned clamped between the jaws of the retractor blades.

FIG. 10 is a cross-sectional view taken along line A-A of FIG. 2 with the ribs positioned clamped between the jaws of the retractor blades that have been separated by rotation of the cog handle to separate the ribs.

FIG. 11 is an end view of one retractor blade assembly.

FIG. 12 is a side view of a retraction blade in the direction of arrow D of FIG. 11.

FIG. 13 is a side view of a retraction blade in the direction of arrow E of FIG. 11.

FIG. 14 is a cross-sectional view taken along line F-F of FIG. 11.

FIG. 15 is an isometric view of the retractor of FIG. 1.

FIGS. 16-19 illustrate a method for attaching the retractor of FIG. 15 to the ribs and then using the retractor to distance the ribs according to the invention.

FIG. 20 illustrates another embodiment of a retractor according to the invention.

FIG. 21 is a detailed view of a cut-away section of the retractor of FIG. 21.

FIG. 22 is a top perspective view of the retractor of FIG. 20 showing rotation of the arm joints.

FIG. 23 is a further view of the retractor of FIG. 22

FIG. 24 illustrates suturing of a pair of ribs following a procedure.

### DETAILED DESCRIPTION OF THE INVENTION

In certain embodiments, the invention describes an exemplary thoracic retractor that is constructed of a pair of retraction members that each comprise a pair of jaws that are movable relative to each other to permit the jaws to grip the anterior and posterior surfaces of the ribs without the retraction members applying a force to or crushing the intercostal nerves. This, in turn, assists in minimizing a patient's post-operative pain. A distancing mechanism may be used to move the retraction members laterally away from each other in order to distance the ribs from each other.

In some cases, the distancing mechanism may include a pair of arms that are configured to be coupled to the retraction members. The arms each have at least one joint that permit the retractor members to move relative to the distance mechanism. As an example, the joints may be configured to rotate or pivot.

The distancing mechanism may be constructed of a rack member having a plurality of teeth and a cog mechanism that engages the teeth and moves relative to the rack member when rotated. The rack member is attached to one of the arms and the cog mechanism is attached to the other arm to permit the retraction members to move laterally away from each other when the cog mechanism is rotated.

Another aspect is the use of an optional guide member that may be coupled to one or both of the jaws. The guide member has a through hole that is adapted to receive a drill bit or a stabilizing pin. In some cases, the guide member may be tubular and be coupled to a portion of one of the retraction members to facilitate placement of a pin into the rib.

The invention also provides exemplary methods for accessing a body cavity. According to one method, two adjacent ribs that are to be separated are located and accessed. A retraction member is attached to each of the ribs. Each retraction member comprises a pair of jaws that are movable relative to each other to permit the jaws to grip the anterior and posterior surfaces of the ribs without the retraction members applying a force to or crushing the intercostal nerves, thus minimizing a patient's post-operative pain. A distancing mechanism is operated to move the retraction members laterally away from each other, thereby distancing the ribs from each other.

In one aspect, a clamping bolt may be rotated to move the jaws relative to each other to permit the jaws to grip the ribs. Also, a hole may be drilled in each rib and a securing pin placed into the holes. Optionally, the pins may be removed from the holes and a suture inserted through the holes to tie the ribs together following a procedure.

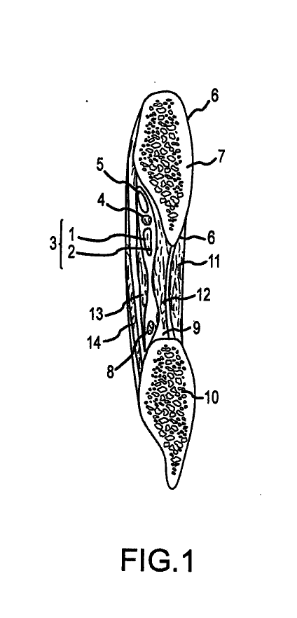

The specific embodiments described below may be used in connection with the anatomy of described in connection with FIG. 1. More specifically, FIG. 1 is a diagram of a vertical section through an intercostal space, showing the relationship of the vessels and nerves to the muscular layers. The close physical relationship of the main intercostal nerve 1 and the external cutaneous nerves 2 have, for clarity in this application, been combined and labeled as a pair and referred to as nerves 3. Adjacent to the nerves 3 are the intercostal artery 4 and vein 5. The nerves 3 protrude beyond the lower lip 6 of the upper rib 7. The collateral branch of the nerve 8 lies adjacent to the upper face 9 of the lower rib 10. Between the upper rib 7 and lower rib 10 lie the external intercostal muscle 11, the internal intercostal muscle 12, the intracostal muscle 13 and the subcostal muscle 14.

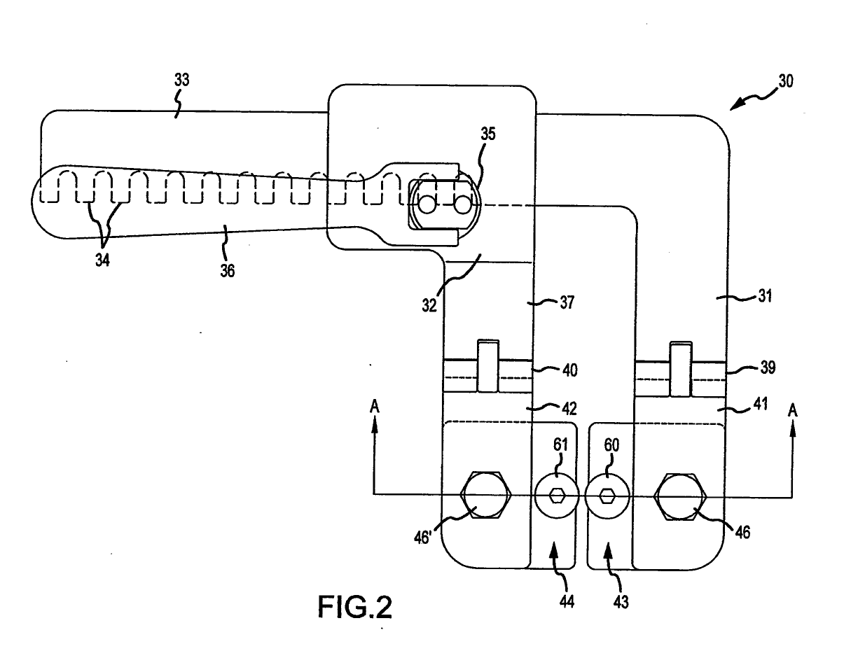

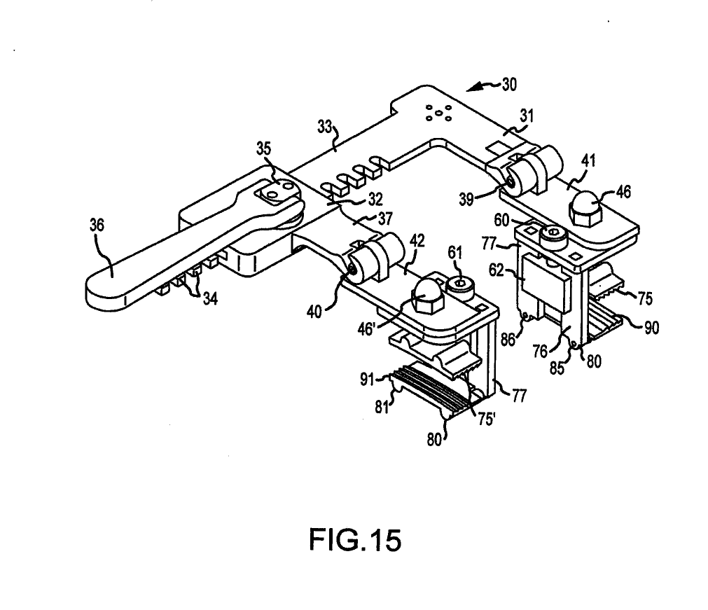

Referring to FIGS. 2, 3 and 15, one embodiment of a retractor that may be used to distance the ribs without unduly interfering with, contacting and/or crushing the nerves mentioned about will be described. FIG. 2 shows a plan view of retractor 30 of this invention with the retraction members in the closed position. The thoracic retractor 30 has a fixed arm 31 joined to a rack member 33 that has a series of teeth 34 between the end of the rack 33. A moving block member 32 has a slot (not shown) of dimensions that allow the member 32 to freely slide along the rack 33. A cog mechanism 35 engages in the moving arm teeth 34 such that rotation of a hinged cog handle 36 drives a moving arm 37 along the rack 33 in the direction depending on the direction of rotation of the cog handle 36 to form a distancing mechanism. The moving arm 37 is attached to moving block 32. Handle 36 is hinged on the cog mechanism 35 by a pin 38. In one embodiment of the invention arms 31 and 37 each terminate in an end that includes a slot that are used to form hinges as described hereinafter. Pivotally attached to the ends of arms 31 and 37 are extension arms 41, 42 that each include hinge pins 39, 40. In turn, hinge pins 39, 40 fit within the slots to permit arms 31 and 37 to rotate about hinge pins 39, 40. Vertically hinged to extensions 41, 42 are blade vice assemblies 43, 44, also referred to as retraction members. Hinge bolts 45, 45′ are clearance fit in plates 50, 51, and threaded into extension arm 41, 42, and retained by acorn nuts 46, 46′.

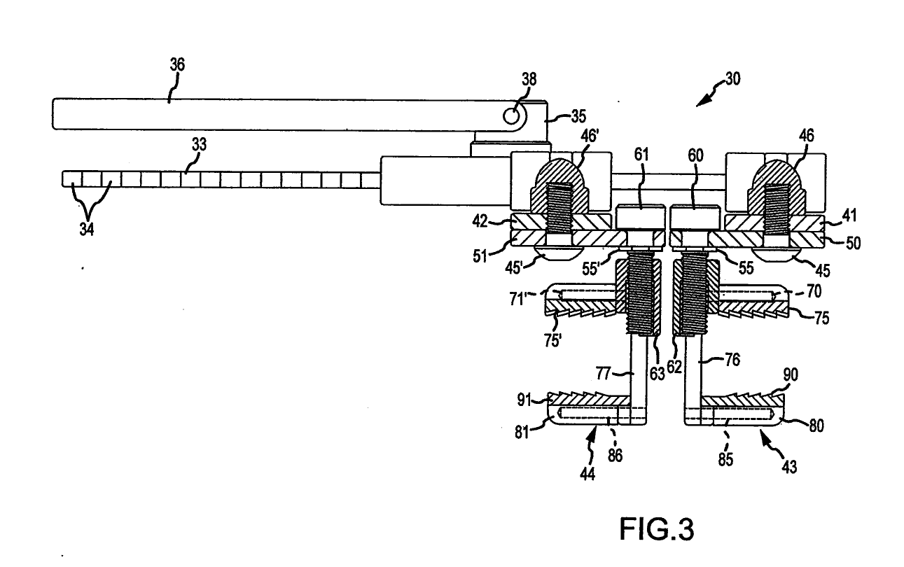

Shown in FIG. 3 are a pair of clamping bolts 60, 61 that are preferably 8-32 left hand thread stainless steel hexagonal head cap screws that are a clearance fit in arm extensions 50, 51. These cap screws are retained by a pair of “E” clip retainers 55, 55′. The cap screws are threaded into sliding blocks 62, 63 so the rotation of a cap screw will cause its associated block to move downwards or upwards depending on the direction the cap screw is turned. The sliding blocks 62, 63 each have a pair of firmly attached protruding stainless steel down pins 70, 71 and 70′, 71′ onto which upper toothed jaws 75, 75′ are pushed and lightly retained such that they may be pulled off, but will not fall off.

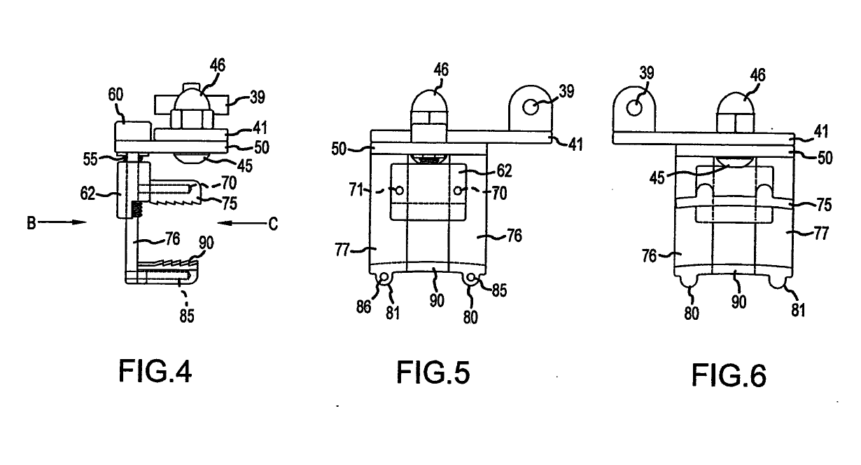

FIG. 4 shows an end view elevation of blade vice assembly 43, it being appreciated that blade vice assembly 44 is constructed in a similar manner and may include some of the same reference numerals. FIG. 5 shows a side of blade vice assembly 43 taken in the direction of arrow B of FIG. 4, and FIG. 6 shows a side of blade vice assembly 43 taken in the direction of arrow C of FIG. 4. A pair of vertical rectangular stainless steel pillars 76, 77 are firmly attached to upper plate 50. The pillars 76, 77 terminate in a semicircular protrusion 80, 81 into which a pair of stainless dowel pins 85, 86 are firmly attached, onto which a lower toothed jaws 90 and 91 are pushed and lightly retained such that it may be pulled off, but will not fall off.

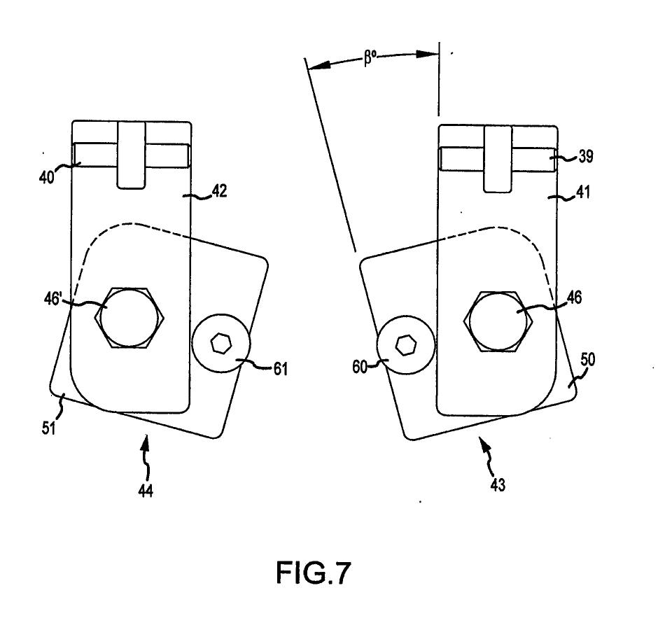

FIG. 7 shows the pivoting of the blade vice assemblies 43, 44 as the ribs are retracted. Cap screws 60, 61 form stops to prevent unwanted free rotation of the blade vice assemblies.

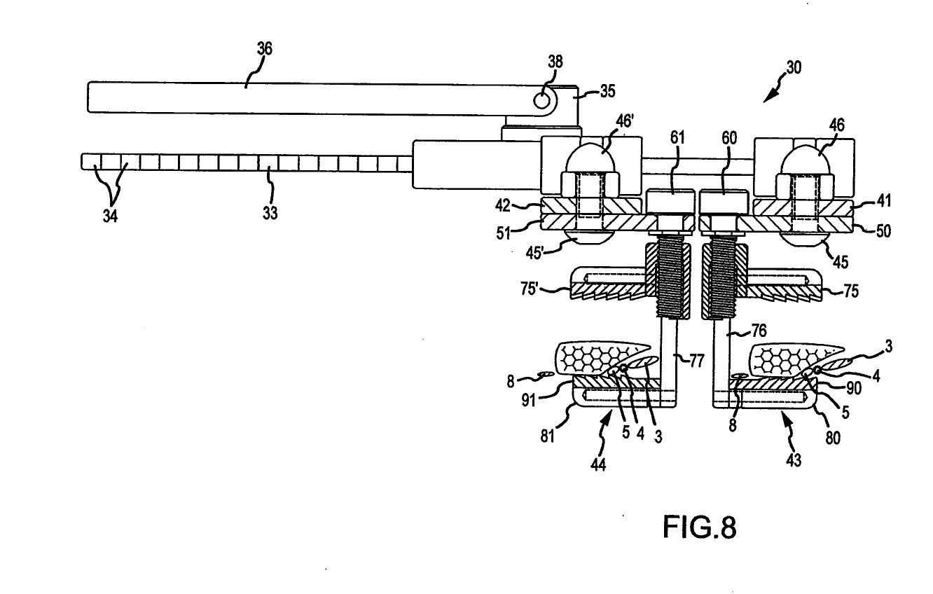

FIG. 8 shows the blade vice assemblies 43, 44 placed under the ribs. Blade vice assemblies 43, 44 are positioned such that they are spaced apart from any of the nerves 3 and 8 (and other vessels) yet still remain above and below the ribs.

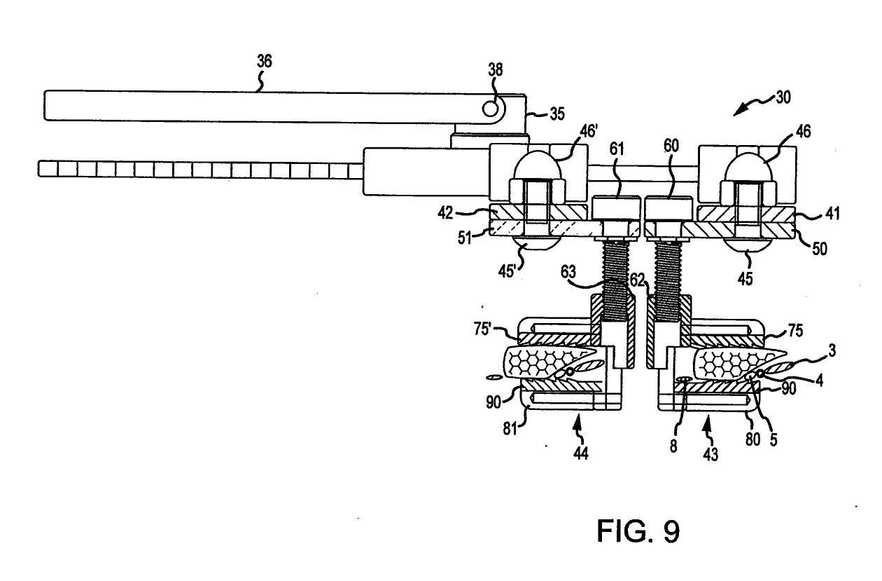

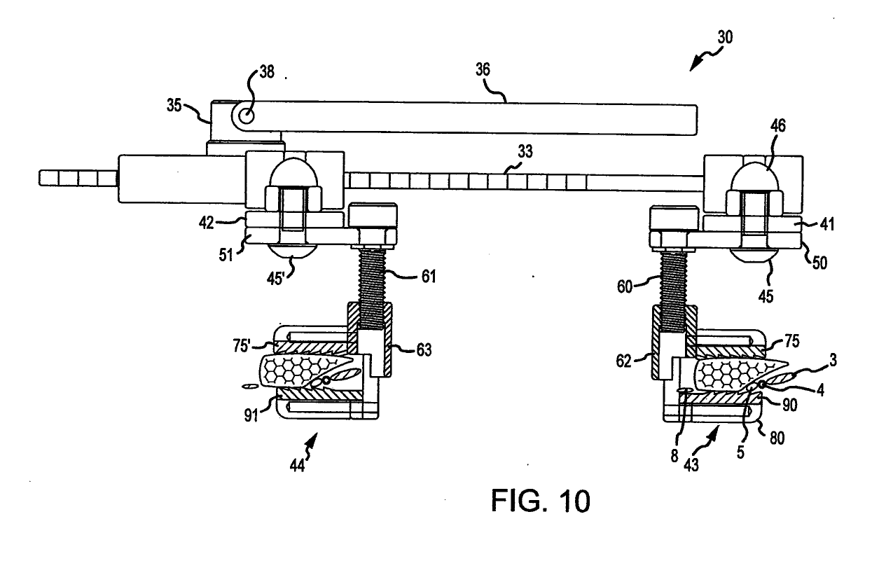

FIG. 9 shows the blade vice assemblies 43, 44 clamping the ribs between the anterior and posterior surfaces of the ribs, and FIG. 10 shows retraction of the ribs to provide surgical access to the inner thorax, without crushing or applied pressure to the nerves 3 and 8.

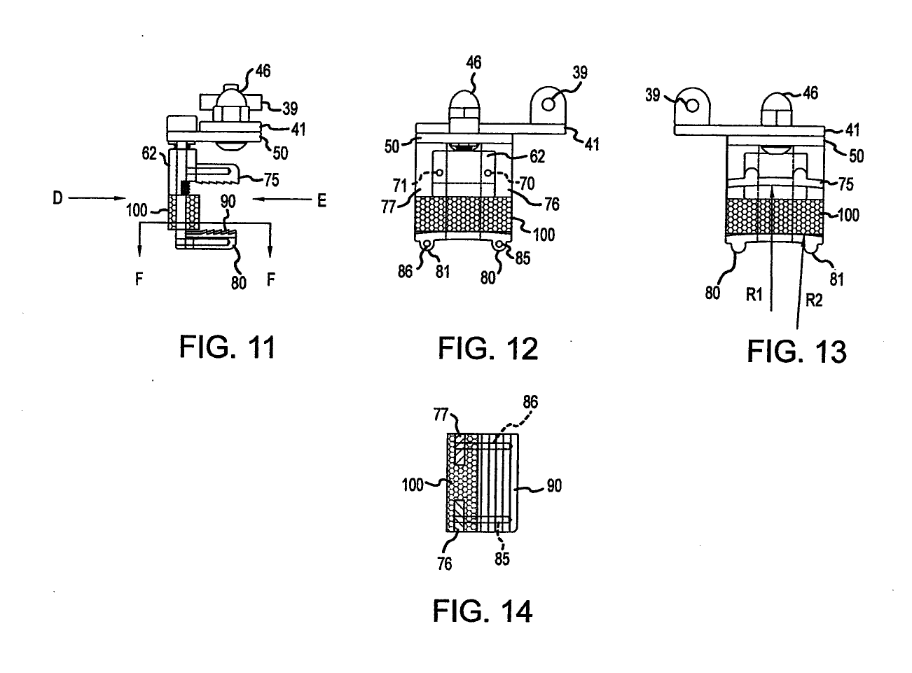

Optionally, one or both of the retractor blade assemblies may include a disposable foam indicator/protection pad. This pad may be used to help position the blade assemblies away from the exposed nerves as well as providing a cushioning protector in the event that the retractors contact the nerves. This pad may be constructed, for example, from a semi-transparent silicone rubber or other foamed material. FIG. 11 shows an end view of one retractor blade vice assembly 43 with a disposable foam indicator/protection pad 100 in position. FIG. 12 shows a side view of a retraction blade in the direction of arrow D of FIG. 11. FIG. 13 shows a side view of a retraction blade in the direction of arrow E of FIG. 11. Also shown in FIG. 13 are the radius R1 and R2 of upper toothed jaw 75 and lower toothed jaw 90, respectively. The dimensions of R1 and R2 are selected so as to generally match the curvature found on the upper and lower surfaces of the human rib. In this way, jaws 75 and 90 will not crush or flatten the rib when the jaws 75 and 90 are secured to the rib. Finally, FIG. 14 shows a cross-sectional view taken along line F-F of FIG. 11.

To attach pad 100, lower jaw 90 may be slid from pins 85 and 86 to permit pad 100 to be slid up along pillars 76. Alternatively, pad 100 could simply be slid in between jaws 75 and 90 from the side. Following a surgical procedure, pad 100 may be discarded while the remainder of the retractor may be cleaned, sterilized and reused with replacement pads 100.

In some embodiments, the retractor blade assemblies, or portions thereof, can be made to be disposable, such as by constructing them of medical grade plastic or a composite. In this way, the assemblies or portions thereof may be discarded after a procedure while the remainder of the retractor may be cleaned, sterilized and reused. Alternatively, the entire retractor could be disposable.

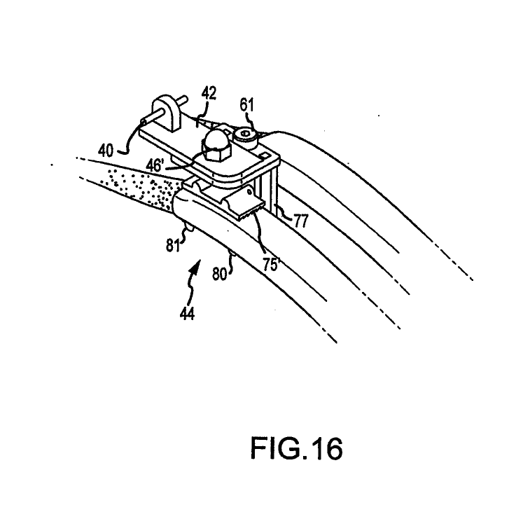

Referring now to FIGS. 16-19, one exemplary method for using retractor 30 to distance a pair of ribs during a surgical procedure will be described. Initially, one or more incisions may be made to gain access to the ribs as is known in the art. Once access has been gained, one of the blade vice assemblies 43 or 44 is attached to one of the ribs. For example, as illustrated in FIG. 16, blade vice assembly 44 is first attached, it being appreciated that blade vice assembly 43 could be attached first as well. To do so, clamping bolt 61 is loosened to insure that upper jaw 75′ is distanced from lower jaw 91 sufficient to permit the jaws to be placed about the rib. Once jaws 75′ and 91 are positioned on the anterior and posterior surfaces of the ribs as shown, clamping bolt 61 may be rotated in order to force the jaws 75′ and 91 toward each other, thereby clamping blade vice assembly 44 securely to the rib. As previously described, the curved surfaces of the upper and lower jaws prevent flattening of the rib when the clamping force is applied. Conveniently, a tool may be used to rotate clamping bolt 61 in order to tighten the jaws.

Importantly, when blade vice assembly 44 is being positioned, stainless steel pillars 76 and 77 will be laterally spaced apart from the rib so as to not interfere with or contact the nerves and veins that run alongside the ribs as previously described. In this way, blade vice assembly 44 is secured to the ribs without contacting, crushing or otherwise interfering with the nerves and veins that run alongside the rib. However, because blade vice assembly 44 is now secured to the rib, a separating force may be applied to the blade vice assembly 44 to separate the ribs without interfering with these nerves and veins.

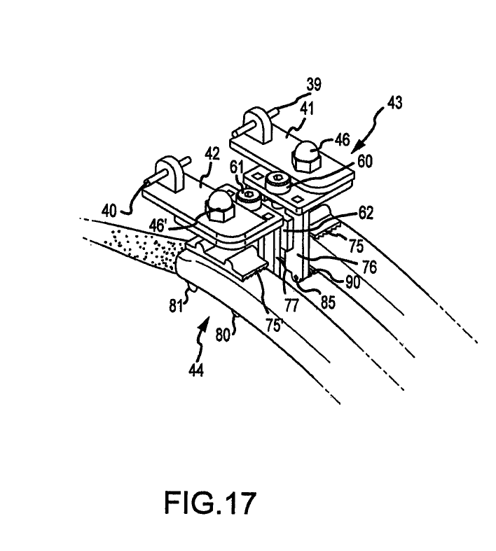

As illustrated in FIG. 17, blade vice assembly 43 is now put in place by being moved over the anterior and posterior surfaces of the adjacent rib in a manner similar to blade vice assembly 44 as just described. When in position, pillars 76 and 77 will be spaced apart from the rib, with jaws 75 and 90 being placed onto the anterior and posterior surfaces of the ribs. Clamping bolt 60 is then rotated to force jaws 75′ and 90 together to securely clamp the adjacent rib without interfering with the nerves or veins.

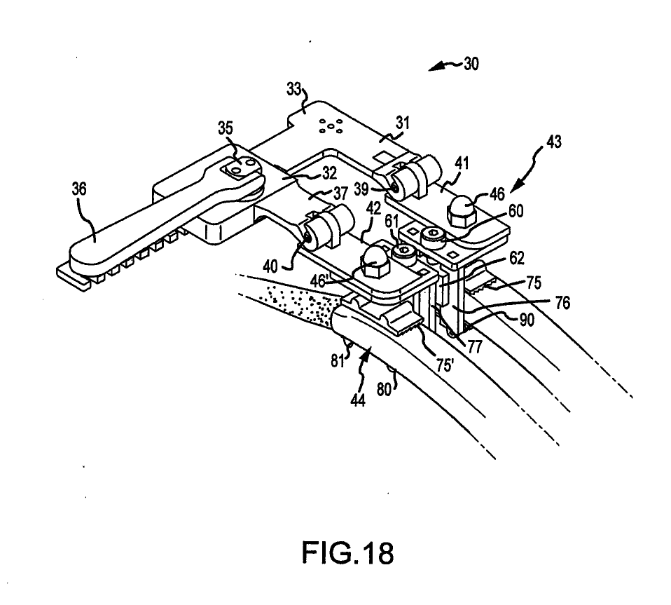

As illustrated in FIG. 18, once both blade vice assemblies 43 and 44 are in place, the remainder of retractor 30 may be assembled. This is done by pivotally attaching the ends of arms 31 and 37 to hinge pins 39 and 40 of extension arms 41 and 42. Because the ends of arms 31 and 37 include slots into which the hinge pins 39 and 40 are received, arms 31 and 37 are able to rotate about the hinge pins 39 and 40. In this way, it is easier for a surgeon to manipulate the position of cog handle 36 when operating retractor 30.

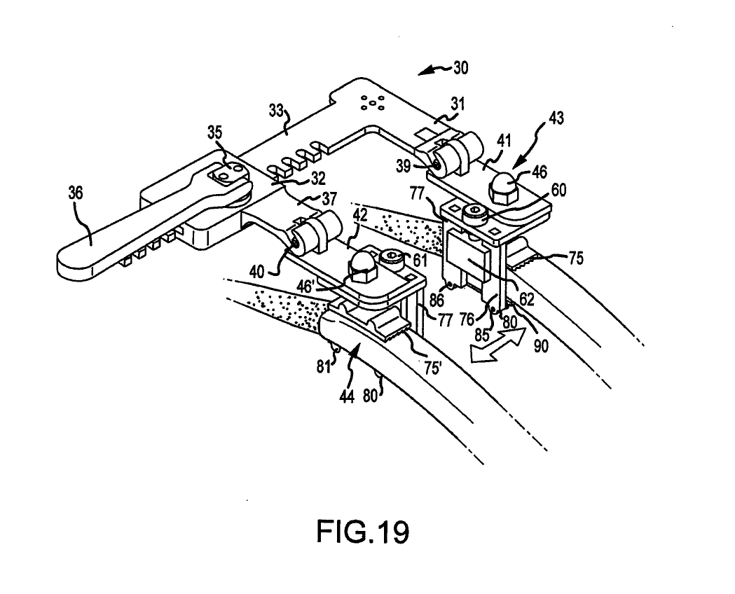

With retractor 30 fully assembled, cog handle 36 may be rotated by the surgeon to distance the two ribs as illustrated in FIG. 19. This is done by simply rotating handle 36 which allows member 32 to slide along rack 33, thereby distancing arms 31 and 37. As arms 31 and 37 move away from each other, some pivoting of extensions 41 and 42 may occur. However, as previously described, the amount of pivoting is limited by bolts 60 and 61. At the same time, blade vice assemblies 43 and 44 are distanced from each other, thereby separating the ribs as shown. However, as previously described, the nerves and arteries extending lengthwise along the ribs are not contacted, crushed or otherwise interfered with so that access to the thoracic cavity may be obtained without causing extensive injury to the patient. Following the procedure, handle 36 may be rotated in the opposite direction until the ribs return to their normal position. At this point, arms 31 and 37 may be removed from hinge pins 39 and 40. At this point, the blade vice assemblies 43 and 44 may be removed by loosening clamping bolts 60 and 61 and slipping the blade vice assemblies 43 and 44 from the ribs.

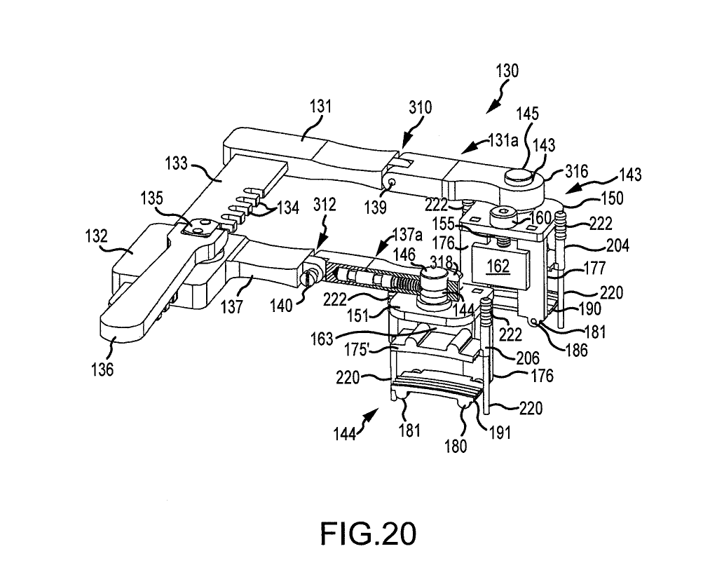

Referring to FIGS. 20-23, another embodiment of a retractor 130 that may be used to distance the ribs will be described. In some aspects, retractor 130 may have components similar to those previously described in connection with retractor 30. For example, thoracic retractor 130 has a fixed arm 131 joined to a rack member 133 that has a series of teeth 134 between the end of the rack 133. A moving block member 132 has a slot (not shown) of dimensions that allow the member 132 to freely slide along the rack 133. A cog mechanism 135 engages in the moving arm teeth 134 such that rotation of a hinged cog handle 136 drives a moving arm 137 along the rack 133 in the direction depending on the direction of rotation of the cog handle 136. The moving arm 137 is attached to moving block 132. Handle 136 is hinged on the cog mechanism 135 to permit handle 136 to pivot, in addition to rotate. Arms 131 and 137 each include at least one joint.

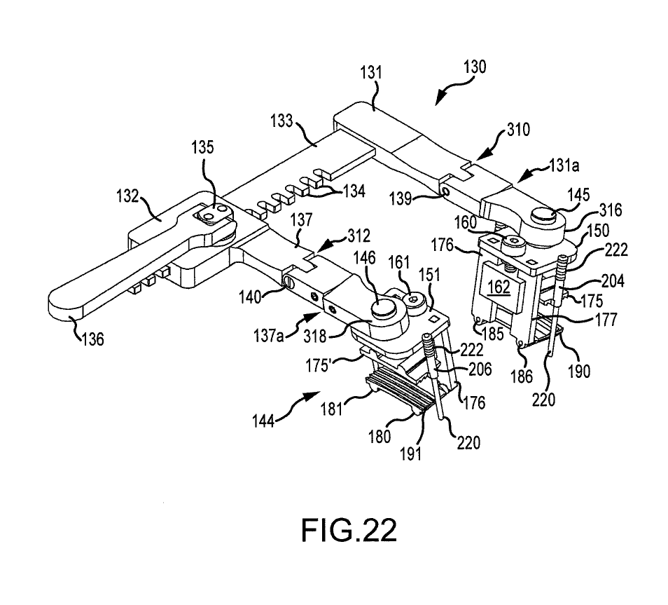

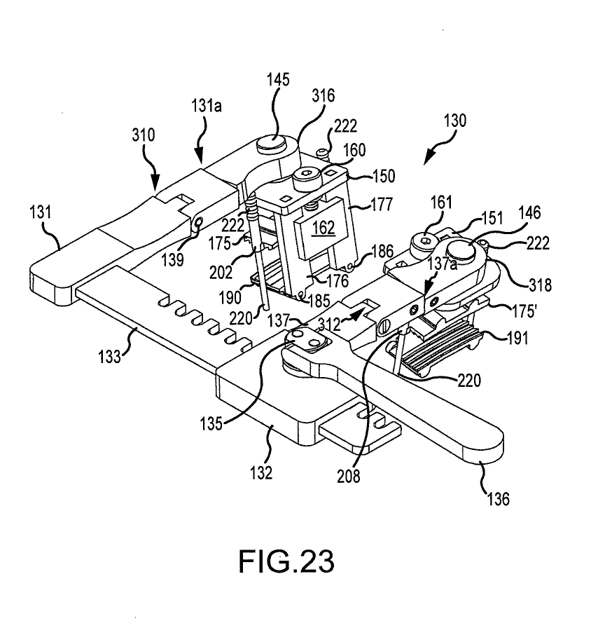

As shown, arms 131 and 137 each include a rotatable joint 131a and 137a that permits the arms to rotate about an axis parallel to rack 133 (see FIGS. 22 and 23) and a hinged joint 310 and 312. In some cases, the two joints could be made as a single ball joint or other articulating member. The use of these joints will be described hereinafter. Arms 131 and 137 each terminate in an end 316 and 318 that includes a through hole 330 and 332 to rotatably connect to the retraction members as described hereinafter.

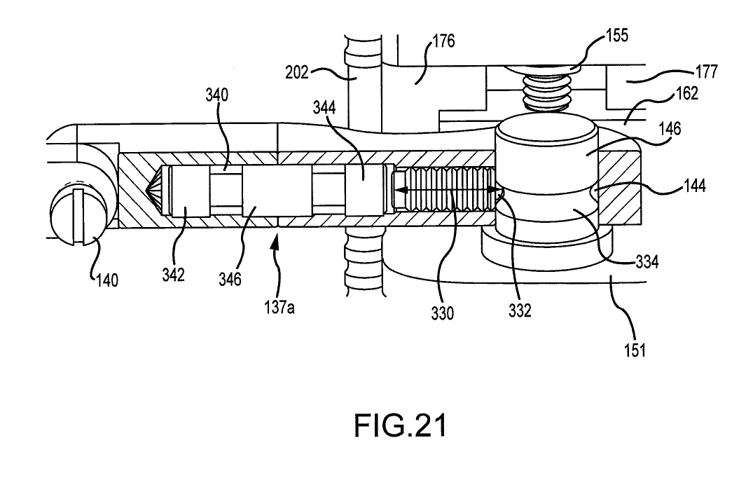

FIG. 21 illustrates construction of joint 137a in greater detail. Joint 137a is formed by providing a cylindrical opening 340 in arm 137 so as to be on both sides of the resulting joint. Two stoppers 342 and 344 are press fit within opening 340 and hold a rotatable member 346 between them. In this way, the resulting joint 137a may rotate 360° about a central axis of arm 137. Some friction may be provided in joint 137a so that a minimal amount of force is required to initiate rotation.

Hinged joints 310 and 312 of arms 131 and 137 include hinge pins 139, 140 that fit within cylindrical openings in arms 131 and 137 to permit arms 131 and 137 to pivot about hinge pins 139, 140.

The ends of arms 131 and 137 are also rotatably connected to blade vice assemblies 143, 144. Cylindrical members 145, 146 extend from plates 150, 151, and are designed to fit within through holes 330 and 332 on the ends of arms 131 and 137. This permits arms 131 and 137 to easily be inserted onto cylindrical members 145 and 146 when attaching the arms during a procedure. Further, arms 131 and 137 may rotate about cylindrical members 330 and 332.

As illustrated in FIG. 21, arm 137 (as well as arm 131) may include a ball plunger 330 having a detent 332 that fits within a groove 334 of cylindrical member 146. Ball plunger 330 fits within opening 340. In this way, blade vice assembly 144 will be securely coupled to arm 137 after it is slipped over cylindrical member 146.

Retractor 130 further includes a pair of clamping bolts 160, 161 (that are preferably 8-32 left hand thread stainless steel hexagonal head cap screws) that are a clearance fit in plates 150, 151. These cap screws are retained by a pair of “E” clip retainers 155. The cap screws are threaded into sliding blocks 162, 163 so the rotation of a cap screw will cause its associated block to move downwards or upwards depending on the direction the cap screw is turned. The sliding blocks 162, 163 each have a pair of firmly attached protruding stainless steel down pins similar to those in other embodiments onto which upper toothed jaws 175, 175′ are pushed and lightly retained such that they may be pulled off, but will not fall off. A pair of vertical rectangular stainless steel pillars 176, 177 are firmly attached to upper plate 150. The pillars 176, 177 terminate in a semicircular protrusion 180, 181 into which a pair of stainless dowel pins 185, 186 are firmly attached, onto which a lower toothed jaws 190 and 191 are pushed and lightly retained such that it may be pulled off, but will not fall off.

Retractor 130 also optionally includes one or more guide members that are used to guide a drill bit when drilling a hole through a rib and/or to hold a pin that extends through the rib. The drilling of such holes and the use of pins as described hereinafter are optional. As shown, coupled to upper jaws 175, 175′ are guide members 202, 204, 206 and 208. These guides may be constructed to have a tubular shape so as to facilitate positioning of a drill bit and/or a pin. For example, pins 220 are shown within guide members 202, 204, 206 and 208. As described below, in practice pins 220 are removed prior to attaching the blade vice assemblies to the ribs. Although shown coupled to the upper jaws, it will be appreciated that other locations may also be used, such as the lower jaws 190, 191 or plates 150, 151. Guide members 202, 204, 206 and 208 may have an internal diameter in the range from about 1.5 mm to about 2 mm and a length in the range from about 12 mm to about 25 mm. Pins 220 each have a head 222 that prevents the pins from slipping through the guide members.

In one method, retractor 130 may be used to facilitate separating the ribs by first making one or more incisions to gain access to the ribs as is known in the art. Once access has been gained, one of the blade vice assemblies 143 or 144 is attached to one of the ribs. To do so, clamping bolt 161 is loosened to insure that upper jaw 175′ is distanced from lower jaw 191 sufficient to permit the jaws to be placed about the rib. Once jaws 175′ and 191 are positioned on the anterior and posterior surfaces of the ribs as shown, clamping bolt 161 may be rotated in order to force the jaws 175′ and 191 toward each other, thereby clamping blade vice assembly 144 securely to the rib. As previously described, the curved surfaces of the upper and lower jaws prevent flattening of the rib when the clamping force is applied. Conveniently, a tool may be used to rotate clamping bolt 161 in order to tighten the jaws.

Importantly, when blade vice assembly 144 is being positioned, stainless steel pillars 176 and 177 will be laterally spaced apart from the rib so as to not interfere with or contact the nerves and veins that run alongside the ribs as previously described. In this way, blade vice assembly 144 is secured to the ribs without contacting, crushing or otherwise interfering with the nerves and veins that run alongside the rib. However, because blade vice assembly 144 is now secured to the rib, a separating force may be applied to the blade vice assembly 144 to separate the ribs without interfering with these nerves and veins.

Blade vice assembly 143 may now be put in place by being moved over the anterior and posterior surfaces of the adjacent rib in a manner similar to blade vice assembly 144 as just described. When in position, pillars 176 and 177 will be spaced apart from the rib, with jaws 175 and 190 being placed onto the anterior and posterior surfaces of the ribs. Clamping bolt 160 is then rotated to force jaws 175′ and 190 together to securely clamp the adjacent rib without interfering with the nerves or veins.

Once at least one of blade vice assemblies 143 and 144 is in place, holes may be drilled into the ribs to facilitate placement of pins. Drilling is accomplished by inserting a drill bit through one of the guide members 202, 204, 206 or 208 and adjacent the rib. The drill bit is rotated until the drill but completely passes through the rib. The drill bit may then be removed and another hole drilled using another one of the guide members. The guide members are positioned such that the holes are drilled generally in the middle of the rib.

Following successful drilling, a pin 220 is placed through each guide member until the pin 220 passes through the rib. Typically the pin head 222 will rest on the guide member to prevent further travel of the pin through the rib. One advantage of using the pins is that they help stabilize the blade vice assemblies so that they do not move or slip off the ribs when the ribs are separated. This permits less force to be applied to the ribs with the jaws. Further, by preventing movement of the blade vice assemblies relative to the ribs, unwanted contact with the nerves is ensured. Still further, because the pins are held in place by the guide members, they may be easily removed when not needed.

With the optional pins in place, the remainder of retractor 130 may be assembled. This is done by pivotally attaching the ends of arms 131 and 137 to cylindrical members 145, 146. Because the ends of arms 131 and 137 include through holes 330, 332 into which the members 145, 146 are received, arms 131 and 137 are able to rotate relative to the blade vice assemblies. In this way, it is easier for the ribs to be separated.

With retractor 130 fully assembled, cog handle 136 may be rotated by the surgeon to distance the two ribs. This is done by simply rotating handle 136 which allows member 132 to slide along rack 133, thereby distancing arms 131 and 137. As arms 131 and 137 move away from each other, arms 131 and 137 may rotate relative to rack 133, particularly about rotatable joints 131a and 137a. Further, because the ends of arms 131 and 137 are also rotatably coupled to cylindrical members 145, 146, the blade vice assemblies may rotate relative to arms 131 and 137. This is important because the two ribs will also rotate and move outwardly as they are separated. If needed, arms 131 and 137 may pivot about hinge pins 139, 140, such as when needing to adjust the position of handle 136.

As previously described, the nerves and arteries extending lengthwise along the ribs are not contacted, crushed or otherwise interfered with so that access to the thoracic cavity may be obtained without causing extensive injury to the patient. Following the procedure, handle 136 may be rotated in the opposite direction until the ribs return to their normal position. At this point, arms 131 and 137 may be removed from blade vice assemblies 143 and 144.

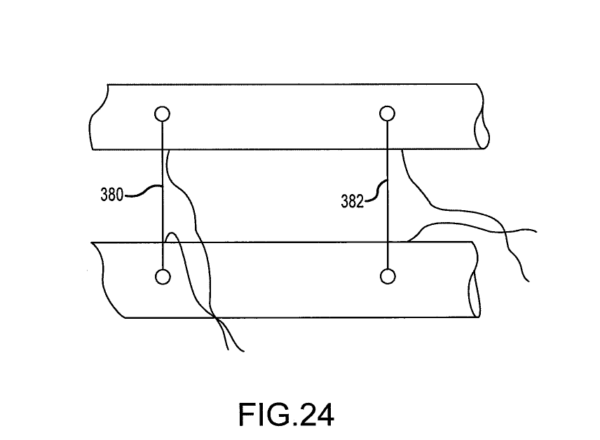

The pins 220 may then be removed from the guide members and the blade vice assemblies 143 and 144 removed from the ribs. Using two of the holes created to hold the pins 220, a length of suture 380, 382 may be inserted through the ribs and used to pull the ribs back together. Preferably, the suture will be inserted from the anterior side of the ribs as shown in FIG. 24. Because the suture passes midway through the ribs, it does not come into contact with the nerves and therefore does not cause excessive trauma when pulling the ribs back together and then suturing the ribs in place.

In an alternative embodiment, a set of pins that extend through the ribs may be used in place of clamps or jaws in order to separate the ribs. These pins could be similar to the pins as previously described, or could be a mechanism that selectively expands at its distal end. Once holes are drilled in the ribs and the pins put in place, a distancing tool may be coupled to the pins. In this way, blade vice assemblies may not be required. The distancing tool may be operated to move the ribs apart using the pins in a manner similar to the other embodiments described herein.

In such a procedure, a pin that expands at its distal end once in place may be used to prevent the pin from slipping out. A mechanism at the proximal end may be operated to release the pin when the operation is completed. One example of such a pin is a Clecos fastener.

The invention has now been described in detail for purposes of clarity and understanding. However, it will be appreciated that certain changes and modifications may be practiced within the scope of the appended claims.

What is claimed is:

1. A thoracic retractor comprising:
a pair of retraction members that each comprise a pair of jaws that are movable relative to each other to permit the jaws to grip the anterior and posterior surfaces of the ribs without the retraction members applying a force to or crushing the intercostal nerves, thus minimizing a patient's post-operative pain; and
a distancing mechanism that is configured to move the retraction members laterally away from each other in order to distance the ribs from each other.

2. A retractor as in claim 1, wherein the distancing mechanism includes a pair of arms that are configured to be coupled to the retraction members, and wherein the arms each have at least one joint that permit the retractor members to move relative to the distance mechanism.

3. A retractor as in claim 2, wherein the joint is configured to rotate or pivot.

4. A retractor as in claim 2, wherein the distancing mechanism further comprises a rack member having a plurality of teeth and a cog mechanism that engages the teeth and moves relative to the rack member when rotated, wherein the rack member is attached to one of the arms and the cog mechanism is attached to the other arm.

5. A thoracic retractor comprising:
at least one retraction member comprising a pair of jaws that are movable relative to each other to permit the jaws to grip the anterior and posterior surfaces of the ribs without the retraction member applying a force to or crushing the intercostal nerves, thus minimizing a patient's post-operative pain; and
a guide member coupled to one of the jaws, the guide member having a through hole that is adapted to receive a drill bit or a stabilizing pin.

6. A retractor as in claim 5, further comprising a second retraction member and a distancing mechanism that is configured to move the retraction members laterally away from each other in order to distance the ribs from each other.

7. A retractor as in claims 6, wherein the distancing mechanism includes a pair of arms that are configured to be coupled to the retraction members, and wherein the arms each have at least one joint that permits the retractor members to move relative to the distancing mechanism.

8. A retractor as in claim 7, wherein the distancing mechanism further comprises a rack member having a plurality of teeth and a cog mechanism that engages the teeth and moves relative to the rack member when rotated, wherein the rack member is attached to one of the arms and the cog mechanism is attached to the other arm.

9. A thoracic retractor comprising:
a pair of retraction members that each comprise a pair of jaws that are movable relative to each other to permit the jaws to grip the anterior and posterior surfaces of the ribs without the retraction members applying a force to or crushing the intercostal nerves, thus minimizing a patient's post-operative pain;
at least one tubular guide member coupled to one of the retraction members to facilitate placement of a pin into the rib; and
a distancing mechanism that is configured to move the retraction members laterally away from each other in order to distance the ribs from each other, wherein the distancing mechanism includes at least one joint to permit relative movement between at least one of the retractor members and the distancing mechanism.

10. A retractor as in claim 9, wherein the distancing mechanism includes a pair of arms that are coupled to the retraction mechanism.

11. A retractor as in claim 10, wherein the distancing mechanism further comprises a rack member having a plurality of teeth and a cog mechanism that engages the teeth and moves relative to the rack member when rotated, wherein the rack member is attached to one of the arms and the cog mechanism is attached to the other arm.

12. A method for accessing a body cavity comprising:
locating two adjacent ribs that are to be separated;
attaching a retraction member to each of the ribs, wherein each retraction member comprises a pair of jaws that are movable relative to each other to permit the jaws to grip the anterior and posterior surfaces of the ribs without the retraction members applying a force to or crushing the intercostal nerves, thus minimizing a patient's post-operative pain;
operating a distancing mechanism to move the retraction members laterally away from each other, thereby distancing the ribs from each other.

13. A method as in claim 12, further comprising rotating a clamping bolt to move the jaws relative to each other to permit the jaws to grip the ribs.

14. A method as in claim 12, further comprising drilling a hole in each rib and placing a securing pin into the holes.

15. A method as in claim 14, further comprising removing the pins from the holes and inserting a suture through the holes to tie the ribs together following a procedure.

16. A method as in claim 12, wherein the distancing mechanism comprises a pair of arms, and further comprising pivotally or rotationally attaching the arms to the retraction members and distancing the arms laterally away from each other.
